(19) United States
(12) Patent Application Publication
(10) Pub. No.: US 2013/0253400 A1
(43) Pub. Date: Sep. 26, 2013

(54) THUMB SUPPORT

(71) Applicant: Gordon W. MASSA, Mason, OH (US)

(72) Inventor: Gordon W. MASSA, Mason, OH (US)

(21) Appl. No.: 13/833,429

(22) Filed: Mar. 15, 2013

Related U.S. Application Data
(60) Provisional application No. 61/614,208, filed on Mar. 22, 2012.

(51) Int. Cl. A61F 13/10

(52) U.S. Cl. CPC A61F 13/105
USPC 602/22

(57) ABSTRACT

A thumb support for preventing injury to the thumb of a user by preventing the thumb from being forcefully hyper-extended or radially abducted while permitting the user to flex or move the thumb towards the palm of the hand in a grasping or pinching motion. The support includes a thumb stabilizing component and a wrap component, and can also prevent wrist injury from hyper-extension. The thumb support typically includes a loop formed at a first end of a strap, the loop being dimensioned to accommodate a user's thumb, near the base thereof, and an elongated portion extending from the loop, of a length sufficient to diagonally follow the metacarpal of the user's thumb and wrap around the user's wrist at least one time.

### CROSS-REFERENCE TO RELATED APPLICATIONS

This application claims the benefit of U.S. Provisional Application 61/614,208, filed Mar. 22, 2012, the disclosure of which is incorporated herein by reference.

### FIELD OF THE INVENTION

The invention relates in general to devices and methods for protecting bones, ligaments and joints of the human hand and wrist, and in particular to a support for preventing injury to the thumb and wrist caused by forced hyper-extension of the thumb or wrist or forced radial abduction of the thumb, while allowing for normal flexion of the thumb.

### BACKGROUND OF THE INVENTION

On the football field, offensive linemen create holes for running backs and protect the quarterback, while defensive linemen rush the passer, tackle the runner and provide the main coverage over the middle of the field. Indeed, linemen use their hands as weapons and, without proper protection, can end up suffering a variety of hand and wrist injuries.

A common football injury to the thumb occurs when the thumb is forcefully hyper-extended or radially abducted while the rest of the hand is moving forward. This can happen during blocking, tackling, or by getting caught in the equipment of another player, such as a jersey, facemask or shoulder pads. The injury may result in broken bones, torn ligaments, weakness, pain, swelling, and tenderness at the inner part of the base of the thumb where the thumb attaches to the hand. The wrist can also suffer hyper-extension injury from forced hyper-extension of the thumb. Linemen, and in particular offensive linemen, are taught to engage and control their opponent by grabbing the chest plate of their shoulder pads with the thumbs up. In this position, any sudden and forceful counter movement by the opponent can cause the thumb to be pulled backwards towards the back of the hand and/or radially away from the index finger towards the arm. When this occurs, forced hyperextension and/or radial abduction of the thumb can cause a sprain or tear of the ulnar collateral ligament, volar plate injury, or damage to the metacarpal phalangeal joint of the thumb.

If surgery is not required to repair a torn ligament or a fracture of the base of the thumb, such sprains may be treated with a cast, taping, or splinting. Casts and splint devices commonly used for the thumb typically utilize a rigid splint which secures the thumb, and sometimes the wrist, in a fixed position. Such rigid and fixed methods of support completely immobilize the thumb. The drawback of taping the thumb is that it takes time to have another person apply the tape. Typically a large amount of tape is required, which must be replaced often. The tape can be cumbersome and immobilizing and at times cut off circulation to the thumb, as well. However, for active players it is generally desirable to support the thumb in a manner which permits movement of the wrist and flexion of the thumb so that a grasping or pinching movement is possible, while preventing hyper-extension and radial abduction of the thumb.

While prior art thumb splint devices may be useful for their intended purposes, they generally do not prevent thumb hyper-extension and radial abduction while allowing flexion of the thumb, or a full grasping motion of the thumb and hand. Further, prior art supports and splints generally obstruct the palm of the hand and thus render hand movement substantially debilitated.

It is, therefore, an objective of the present invention to provide a thumb support which prevents forced thumb hyper-extension and radial abduction, yet allows for normal flexion of the thumb to allow a grasping or pinching motion. It is also an objective to provide a support for the thumb that prevents thumb volar plate injury and protects the metacarpal phalangeal joint of the thumb, along with preventing wrist hyper-extension

### SUMMARY OF THE INVENTION

The present invention is a device and method for preventing injury to the thumb and wrist caused by forced hyper-extension and radial abduction of the thumb, while protecting the metacarpal phalangeal joint of the thumb. The support of the invention protects the thumb and wrist from injury while allowing substantially normal flexing and grasping motion thereof. The support works without rigid components, and yet is low in cost, lightweight, comfortable, easy to apply and/or remove, and easy to manufacture.

A first aspect of the invention relates to a thumb support comprising a stabilizing component for preventing injury to a user's thumb by limiting the thumb from being forcefully hyper-extended or radially abducted while permitting the user to move the thumb towards the palm of the hand; and a wrap component for wrapping around the wrist and securing the thumb stabilizing component in proper position on the hand of the user.

A second aspect of the invention relates to a thumb support for preventing injury to a user's thumb by limiting hyper-extension and radial abduction of the thumb, the support comprising: (a) a loop formed at a first end of a strap, the loop being dimensioned to accommodate a user's thumb, near the base thereof; and (b) an elongated portion of the strap extending from the loop, of a length sufficient to diagonally follow the metacarpal of the user's thumb and wrap around the user's wrist at least one time, wherein the support limits thumb and wrist hyper-extension and radial abduction of the thumb while permitting the user to move the thumb and hand in a grasping or pinching motion.

A third aspect of the invention relates to a method of securing a thumb support to a thumb and wrist of a human hand, the method comprising the steps of: (a) providing a thumb support for preventing injury to a user's thumb by limiting hyper-extension and radial abduction of the thumb; (b) holding the hand in a cupping position; (c) anchoring the thumb support onto the base of the thumb while maintaining the hand in the cupping position; and (d) securing the thumb support around the hand and wrist while maintaining the hand in the cupping position, wherein securing in this manner prevents injury to the user's thumb by limiting forced hyper-extension and radial abduction of the thumb while permitting the thumb and hand to move in a grasping or pinching motion.

A further understanding of the nature and advantages of the invention will be more fully appreciated with respect to the following drawings and detailed description.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the principles of the invention.

FIG. 1A and 1B schematically illustrates the components of the system of the present invention and its mode of interaction.

FIG. 2 is an illustration of the desired “cupping” hand position for applying the support of the invention.

FIGS. 3A-3D provide a sequential view of the palmar and dorsal sides of the hand for illustrating placement of the device around the thumb and wrist.

### DETAILED DESCRIPTION OF THE INVENTION

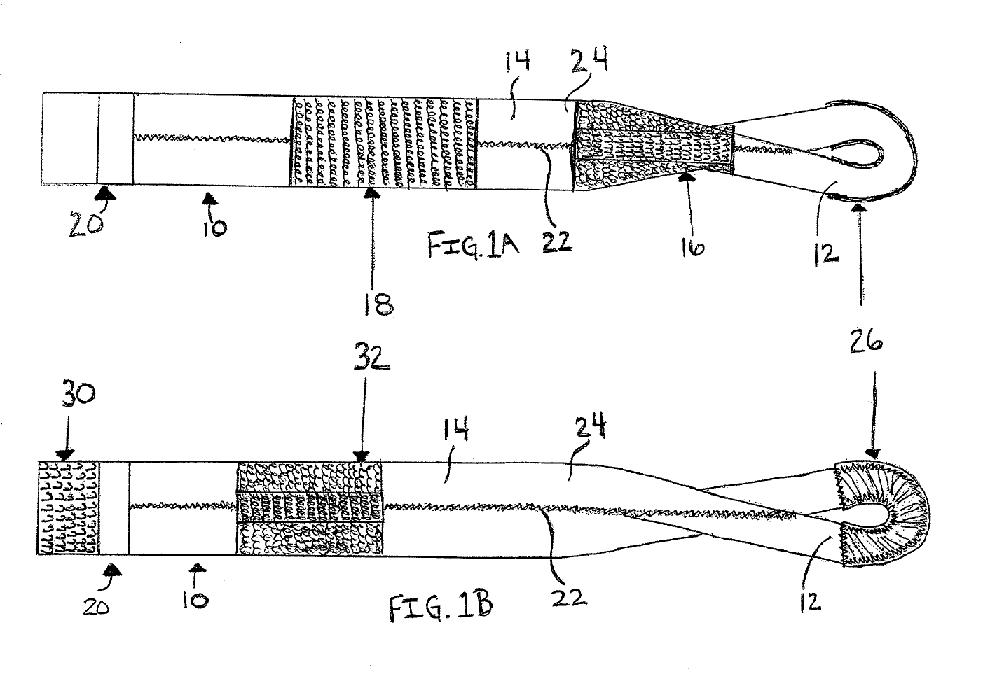

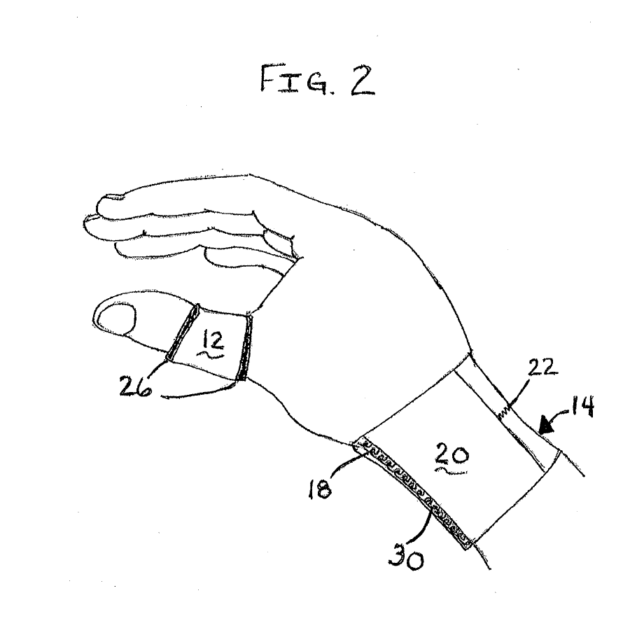

The present invention is a thumb support as illustrated in FIGS. 1A and 1B. The support 10 is generally in the form of a strap which includes a thumb stabilizing component 12 at one end, in the form of a loop, and an elongated wrap component 14 which wraps around the base of the hand and the wrist and secures the thumb stabilizing component 12 in proper position on the hand of the user.

More specifically, FIG. 1A illustrates a “top” portion of the support 10 as it looks prior to insertion onto the hand. The top portion includes fixation means 16 and 18, typically in the form of a hook and loop or Velcro® material. An elastic or stretchable portion 20 is included at the end of the strap 10 for stretching the end of the strap around the wrist and creating a secure fit. As seen in FIG. 1B, the “back” portion of the support 10 also includes a fixation means 30 on the opposite side of the stretchable portion 20, and also fixation means 32 is placed to mate with or otherwise releasably engage fixation means 16 as the elongated wrap component 14 is applied to the wrist.

Still looking at FIGS. 1A and 1B, it can be appreciated that the majority of the support 10 is comprised of a single piece of material 24 in which the ends have been laid side by side and sewn together at sewing lines 22 after creating the loop portion 12. The loop portion 12 is typically lined with a material 26 such as neoprene or velvet, for comfort. The support material 24 is typically made of cotton, polyester, nylon or a mixture of these materials. The fixation means 16, 18, 30 and 32 can be made of a strip of material having a hook and loop pattern, and these strips of material are sewn or otherwise secured to the main support material 24 of the strap 10. For example, Velcro strips can be fashioned to serve as fixation means 16 and 32, and 18 and 30. It will be understood that various other releasable connectors may be employed as fixation means, including buttons, snaps, and the like.

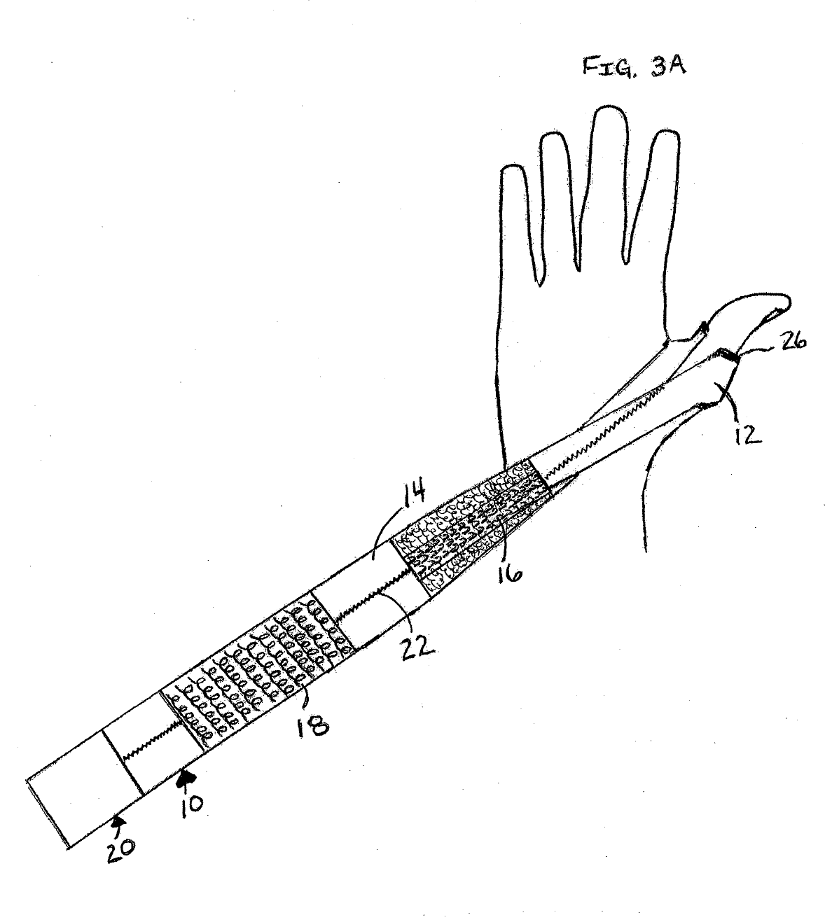

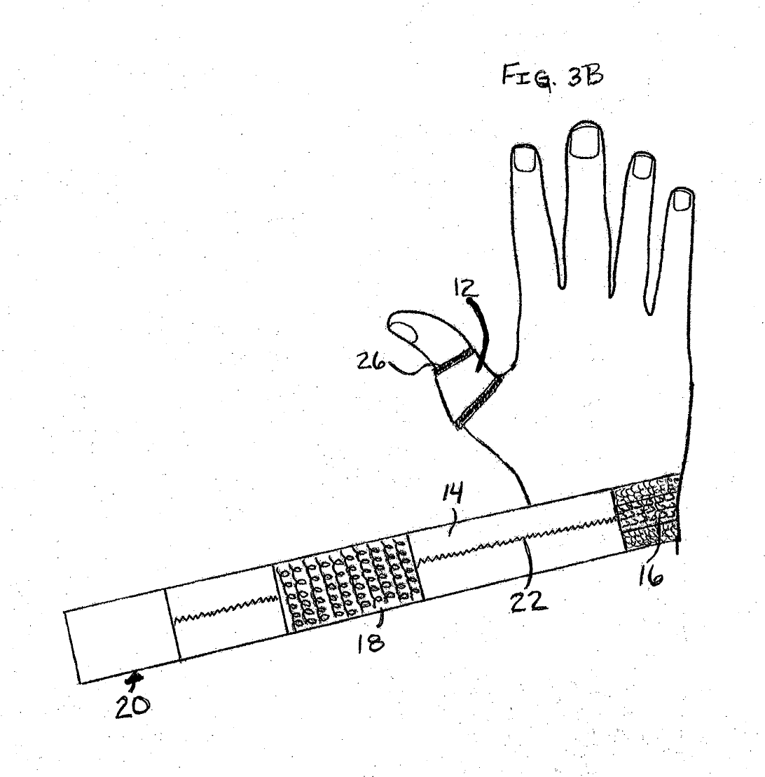

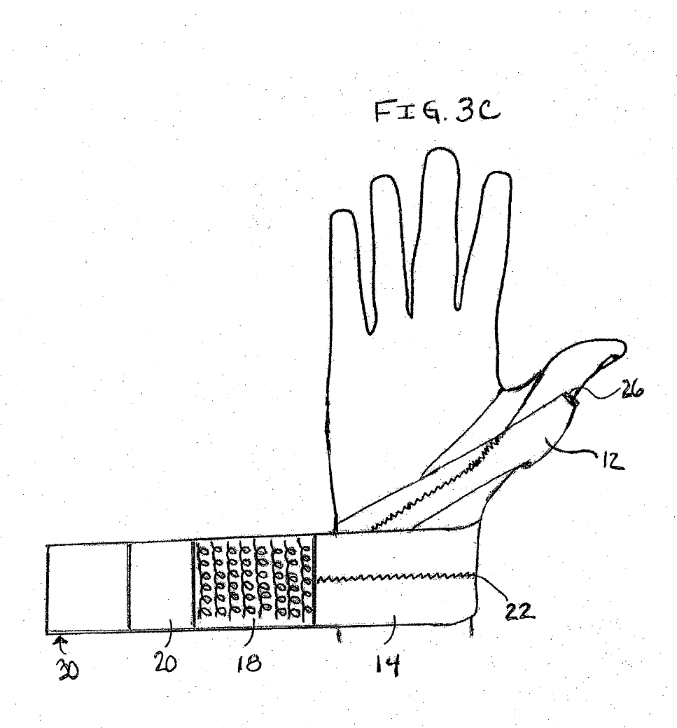

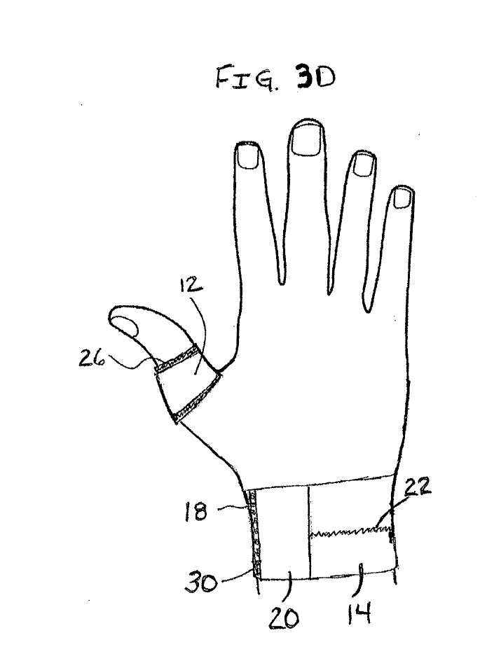

FIG. 2 illustrates the “cupping” position that the hand should be placed in for proper fitting of the support onto the hand, which can be described as the hand position taken when the user is holding a cup, or a can of cola. The user initially holds their hand in this cupping position and then places the loop 12 onto the base of the thumb as an anchor. The elongated portion 14 is then pulled downwardly and diagonally following the metacarpal of the thumb toward the wrist (see FIG. 3A), and then pulled around the back of the hand and wrist (see FIG. 3B), all while maintaining the cupping position. It is important to note that, for proper application of the support of the invention, the hand should be placed in this cupping position as shown in FIG. 2 to ensure proper positioning of the device for preventing injury to the thumb and wrist by forced hyper-extension of the thumb or wrist, or forced radial abduction of the thumb.

FIGS. 3A-3D illustrate a sequential view of the palmar and dorsal sides of the hand for illustrating placement of the device around the thumb and wrist. Please note that it can be appreciated by viewing FIGS. 3A-3D that the hand is not drawn in the cupping position as shown in FIG. 2; however, FIGS. 3A-3D are included to primarily illustrate how the support is wrapped around the anatomy of the hand. To apply the support 10, the thumb stabilizing component 12 is initially placed over the base of the thumb, and the elongated wrap component 14 is then pulled downwardly and diagonally, following the metacarpal of the thumb toward the wrist, see FIG. 3A. The support is then pulled around the back of the hand and wrist, all while the user maintains their hand in a cupping position (not shown). As seen in FIG. 3B, fixation means 16 wraps around the ulnar side of the wrist at the base of the thumb, and as the support encircles the wrist, fixation means 32 will mate with fixation means 16, securing a snug fit around the wrist. As shown in FIG. 3D, fixation means 18 then mates with fixation means 30 as the wrap component 14 completes a full rotation around the wrist. The fixation means 16, 18, 30, 32 serve to help secure a firm fit of the wrap in the proper position, the proper position being achieved when the secured support limits hyper-extension and/or radial extension of the thumb while permitting the thumb and hand to move in a grasping or pinching action or motion.

When the support is properly applied, it can limit the thumb of the user from being voluntarily extended (and also from being forcefully hyper-extended) and radially abducted, while allowing the fingers of the hand to be fully extended. The limitation on voluntary extension or forced hyperextension of the thumb is preferably to a degree sufficient to prevent injury to the thumb. The support may altogether prevent the thumb of the user from being forcefully hyper-extended and radially abducted, while also allowing for thumb flexion, or movement of the thumb towards the palm of the hand, while preventing wrist hyper-extension. Typically the user can still grasp with the hand, enough to form a first with the fingers and thumb, but the thumb cannot be extended backwards.

As noted above, the thumb support 10 of the invention limits or prevents hyper-extension and/or radial abduction of a user's thumb while permitting the thumb, wrist and hand to move in a grasping or pinching action. Typically the loop 12 is formed at a first end of the support and is dimensioned to accommodate the base of the user's thumb. When properly applied by cupping the hand while wrapping the support around the wrist, the loop or thumb stabilizing component 12 in combination with the elongated wrap component 14 precludes the thumb from moving backwards or from being forcefully moved backwards, thereby limiting or preventing hyperextension of the thumb, and also limits or prevents radial extension of the thumb away from the index finger and towards the wrist, while permitting the user to move the thumb towards the palm in a flexing, pinching or grasping motion.

Typically the elongated wrap portion 14 has a width in the range of 2 to 3 inches, and the loop 12 has a width in the range of 0.5 to 1 inch. The support 10 is preferably of sufficient length so as to extend from the loop portion 12, along the metacarpal of the thumb, and wrap around the wrist at least one time, or between about 20 inches to about 40 inches in length. The longer lengths of the support may be desired by athletes who would like the support to wrap around the wrist more than once. The stretchable portion 20 of the support is typically between about 2 inches to 4 inches in length.

For increased appeal, the thumb support is light and thin so that it is comfortable to wear. The support should not interfere with the wearer's ability to move his thumbs or fingers, and can still move the hand in a grasping or pinching motion. The support could be worn by itself or over/under a pair of gloves. It will also be appreciated that the support is quick and easy to put on or take off, and is readily adjustable for a comfortable fit. The support can fit snugly, but does not restrict the flow of blood to the fingers or thumbs, and it does not cause the wearer's hands to sweat excessively.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative system and method, and illustrated examples shown and described. Accordingly, departures may be made from such details without departing from the scope of the invention.

What is claimed is:

1. A thumb support, comprising:
a. a stabilizing component for preventing injury to a user's thumb by limiting the thumb from being forcefully hyper-extended or radially abducted while permitting the user to move the thumb towards the palm of the hand; and
b. a wrap component for wrapping around the wrist and securing the thumb stabilizing component in proper position on the hand of the user.

2. A thumb support for preventing injury to a user's thumb by limiting hyper-extension and radial abduction of the thumb, the support comprising:
a. a loop formed at a first end of a strap, the loop being dimensioned to accommodate a user's thumb, near the base thereof; and
b. an elongated portion of the strap extending from the loop, of a length sufficient to diagonally follow the metacarpal of the user's thumb and wrap around the user's wrist at least one time, wherein the support limits thumb and wrist hyper-extension and radial abduction of the thumb while permitting the user to move the thumb and hand in a grasping or pinching motion.

3. The thumb support of claim 2, wherein the support is made of cotton, polyester, nylon, or a combination thereof.

4. The thumb support of claim 2, wherein the loop includes an elastomeric material.

5. The thumb support of claim 2, further comprising fixation means on the elongated portion of the strap to permit the elongated portion to be affixed around a user's wrist.

6. The thumb support of claim 5, wherein the fixation means comprises a loop/hook material.

7. The thumb support of claim 5, wherein the fixation means comprises a strip of material secured to the strap.

8. The device of claim 1, wherein the elongated strap has a width in the range of 2 to 3 inches, and the loop has a width in the range of 0.5 to 1 inch.

9. A method of securing a thumb support to a thumb and wrist of a human hand, the method comprising the steps of:
a) providing a thumb support for preventing injury to a user's thumb by limiting hyper-extension and radial abduction of the thumb;
b) holding the hand in a cupping position;
c) anchoring the thumb support onto the base of the thumb while maintaining the hand in the cupping position; and
d) securing the thumb support around the hand and wrist while maintaining the hand in the cupping position, wherein securing in this manner prevents injury to the user's thumb by limiting forced hyper-extension and radial abduction of the thumb while permitting the thumb and hand to move in a grasping or pinching motion.

10. The method of claim 9, wherein the thumb support comprises:
a. a loop formed at a first end of a strap, the loop being dimensioned to accommodate a user's thumb, near the base thereof; and
b. an elongated portion of the strap extending from the loop, of a length sufficient to diagonally follow the metacarpal of the user's thumb and wrap around the user's wrist at least one time.

11. The method of claim 10, wherein securing the elongated portion around the hand and wrist is accomplished by wrapping the elongated portion around the wrist such that at least one full wrap around the wrist is made.
